(19) United States
(12) Patent Application Publication
(10) Pub. No.: US 2024/0116886 A1
(43) Pub. Date: Apr. 11, 2024

(54) PROCESS FOR PRODUCING FURFURAL FROM BIOMASS

(71) Applicant: UOP LLC, Des Plaines, IL (US)

(72) Inventors: Phuong T.M. Do, Mount Prospect, IL (US); Anton N. Mlinar, Vernon Hills, IL (US); F. Stephen Lupton, Evanston, IL (US); Hani Gadalla, Palatine, IL (US); Kerry S. Crosby, Mount Prospect, IL (US)

(21) Appl. No.: 18/325,264

(22) Filed: May 30, 2023

Related U.S. Application Data
(60) Provisional application No. 63/377,728, filed on Sep. 30, 2022.

(51) Int. Cl. C07D 307/50

(52) U.S. Cl. CPC C07D 307/50

(57) ABSTRACT

A process for making furfural, derivatives of furfural, or both from biomass is described. The biomass feed is optionally conditioned to obtain a moisture content of 25 wt % or less. The biomass feed is ground to form particles having a particle size in a range of 0.02 mm to 7 mm with 5 wt % or less being 75 microns or less, and an average aspect ratio of 0.4 or more. The ground biomass feed is blanketed with an inert gas having an oxidant or oxygen content of 5 mol % or less. The ground biomass feed is reacted in the presence of water, a water-immiscible organic solvent, and an acid catalyst to form a reaction product mixture comprising furfural, derivatives of furfural, or both.

### RELATED APPLICATIONS

This application claims priority to U.S. Provisional Patent Application Ser. No. 63/377,728 filed on Sep. 30, 2022, the entirety of which is incorporated herein by reference.

### BACKGROUND

Furfural is commercially produced from lignocellulose found in various herbaceous and woody biomass sources. Lignocellulose is the structural component of plant cell walls and is a complex mix of three main components: cellulose, hemicellulose, and lignin.

Cellulose is a linear chain of several hundred to many thousands of β linked D-glucose units. Hemicellulose is a heteropolymer, arabinoxylan, present along with cellulose in almost all terrestrial plant cell walls. While cellulose is crystalline, strong, and resistant to hydrolysis, hemicelluloses have random, amorphous structures with little strength. Lignin is a polymeric material that is a cross-linked component of three monolignols: coumaroyl alcohol, coniferyl alcohol, and sinapyl alcohol. It is thus a highly aromatic polymer. The different polymers exhibit different reactivity to thermal, chemical, and biological processing.

The traditional process for producing furfural from biomass involves hot acid digestion to hydrolyze the hemicellulose to release the C₅ sugars followed by acid catalyzed isomerization and dehydration of the C₅ sugars to furfural. However, many side reactions can occur due to the complex structure of lignocellulose and the acidic environment. In addition to isomerization and dehydration of the C₅ sugars, a similar process can isomerize the C₆ sugar component of hemicellulose or perhaps even some C₆ sugars from cellulose to 5-hydroxymethylfurfural (5-HMF) which may undergo further hydrolysis to levulinic and formic acids. Acetic acid is also generated from the acetyl groups on the hemicellulose. Furthermore, both furfural and 5-hydroxymethylfurfural can also undergo subsequent polymerization to form humins which are highly crosslinked chains of furan and hexose rings.

However, the yields for commercial processes of producing furfural are only about 50% of theoretical, leading to high production costs.

Therefore, there is a need for improved processes for making furfural from biomass.

### BRIEF DESCRIPTION OF THE DRAWINGS

FIG. 1 is an illustration of one embodiment of a process for furfural from biomass.

### DETAILED DESCRIPTION

The present process involves appropriate selection and pre-treatment of the biomass before the hydrolysis and dehydration reactions. By the proper selection of the acid catalyst, solvent, and reaction conditions, the yield of furfural, derivatives of furfural. or both can be increased to 80%, leading to more economically attractive process. Derivatives of furfural include. but are not limited to, 5-hydroxymethylfurfural, and 5-halogen methylfurfural wherein the halogen comprises a chloride, a bromide, a fluoride, or an iodide moiety.

The first step is selecting a biomass feed. Furfural is a 5-carbon product. The components of lignocellulose which are direct precursors to furfural are the 5-carbon sugars, such as xylose and arabinose, found in the hemicellulose which contains about 80% C₅ sugars. Therefore, biomass that is high in hemicellulose and low in lignin is a desirable feedstock for furfural production. Herbaceous materials tend to be richest in hemicellulose whereas trees, especially softwoods, have low amounts of hemicellulose and higher amounts of lignin. Examples of biomass with high levels of hemicellulose and low levels of lignin include, corn cobs, corn stover, corn bran, oat husks, and wheat straw. Commercial furfural processes use corn cobs and sugar bagasse because they are rich in C₅ sugars and are abundant agricultural wastes. However, other sources of biomass can also be used.

Handling biomass feeds involves a problem which is not present in traditional refinery operations: combustible dust. The dust problem requires special procedures to avoid explosion and/or fire in the solid feeding system to the reactor. The physical specifications of the biomass, including particle size, particle size distribution, and moisture content need to be controlled. Furthermore, given the combustible dust hazard, the oxidant or oxygen content in the biomass conversion process should also be controlled.

The initial moisture content of the raw biomass may be up to about 30 wt % or more. The desired moisture content for the process involves balancing mitigating the risk of combustible dust with the desire to minimize the amount of moisture in the biomass feed going to the reactor. The moisture content of the biomass for the process is typically 25 wt % or less, or 20 wt % or less, or 15 wt % or less, or 5 wt % to 15 wt %. As a result, the raw biomass may need to be conditioned to obtain the desired moisture content. Typically, the conditioning will involve drying the raw biomass. In other cases, the moisture content of the biomass may need to be increased using humidification.

The particle size and shape of the biomass feed sent to the reactor should also be controlled. The biomass is ground to obtain a ground biomass feed having an average particle size in the range of 0.02 mm to 10 mm, or 0.02 mm to 9 mm, or 0.02 mm to 8 mm, or 0.02 mm to 7 mm, or 0.02 mm to 6 mm, or 0.02 mm to 5 mm, or 0.02 mm to 4 mm, or 0.02 mm to 3 mm, or 0.02 mm to 2 mm, or 0.02 mm to 1 mm. In addition, the ground biomass feed should have 5 wt % or less of particles having a size of 75 microns or less, or 3 wt % or less, or 1 wt % or less. Ground biomass particles come in a variety of shapes, e.g., round, elongated, irregular, etc., which influences the aspect ratio. Lower aspect ratios correspond to higher concentrations of irregular shapes. These irregular shaped particles pose a problem for reactor internals and downstream equipment. Therefore, the aspect ratio of the ground biomass feed should be controlled to 0.4 or more, or 0.45 or more, or 0.5 or more, or 0.55 or more, or 0.6 or more, or in the range of 0.4 to 1.0, or 0.45 to 1.0, or 0.5 to 1.0, or 0.55 to 1.0, or 0.6 to 1.0.

The ground biomass feed should be blanketed with an inert gas having 5 mol % or less of an oxidizing agent or oxygen gas, or 3 mol % or less, or 1 mol % or less. This helps to reduce the rink of explosion and/or fire. Limiting the amount of oxidant and/or oxygen also reduces the furfural oligomerization/polymerization side reactions which can occur after their formation in the reactor, which leads to loss of yield as well as fouling in downstream separation process, such as fractionation.

The ground biomass feed can be contained in a collection tank with the inert gas blanket before being sent to the reactor.

The reactor contains the ground biomass feed, a solvent, and an acid catalyst.

The solvent may comprise water and/or a water-immiscible organic solvent. The use of a water-immiscible organic solvent improves the furfural yield by extracting the furfural from the aqueous acidic environment in the reactor. By extracting the furfural as it is formed, the opportunity for it to react with other furfural derivatives and biomass byproducts while in the presence of the acid catalyst is greatly reduced. Suitable water-immiscible organic solvents include, but are not limited to, aromatic hydrocarbons and alkyl ketones. Suitable aromatic hydrocarbons include, but are not limited to, benzene, toluene, a xylene, tetralin, an alkyl tetralin, an alkyl naphthalene, an aromatic alcohol, an alkyl phenol, or combinations thereof. Suitable alkyl ketones include, but are not limited to, methyl isobutyl ketone.

The reactions are catalyzed using an acid catalyst. Any suitable acid catalyst may be used. The acid catalyst may comprise a mineral acid, a solid acid, an organic acid, or combinations thereof. Suitable mineral acids include, but are not limited to, sulfuric acid, hydrochloric acid, nitric acid, or combinations thereof. Suitable solid acids include, but are not limited to, AlCl₃, CrCl₃, CrCl₂, acidic zeolite, or combinations thereof. Suitable organic acids include, but are not limited to, carboxylic acids, such as acetic acid, formic acid, propanoic acid, butyric acid, or combinations thereof. Longer chain carboxylic acids could also be used.

The hydrolysis reaction involves the hydrolysis of bound C₅ sugars in the biomass to produce xylose. The xylose is then dehydrated to produce furfural and/or furfural derivatives either in a separate reactor or the same reactor as the hydrolysis reaction.

The process may utilize a single reactor for both the hydrolysis and dehydration reactions. Alternatively, there may be two (or more) reactors, which can be arranged in series or in parallel. The oxidant and/or oxygen content in the reactor(s) should be controlled to prevent subsequent furfural reactions initiated by oxygen leading to decreased furfural yield. The oxidant and/or oxygen content in the reactor(s) should be 5 mol % or less of an oxidizing agent or oxygen gas, or 3 mol % or less, or 1 mol % or less.

Typical reaction conditions for a single reactor include a temperature in the range of 50° C. to 300° C., pressures in the range of 0.3 MPa to 35 MPa, and reaction times in the range of 10 minutes to 60 hours.

In a two (or more) reactor process, typical reaction conditions for the hydrolysis and dehydrogenation reactors include a temperature in the range of 50° C. to 300° C., pressures in the range of 0.3 MPa to 35 MPa, and reaction times in the range of 10 minutes to 60 hours. The reactors may utilize the same catalysts, or the catalysts can be different in different reactors.

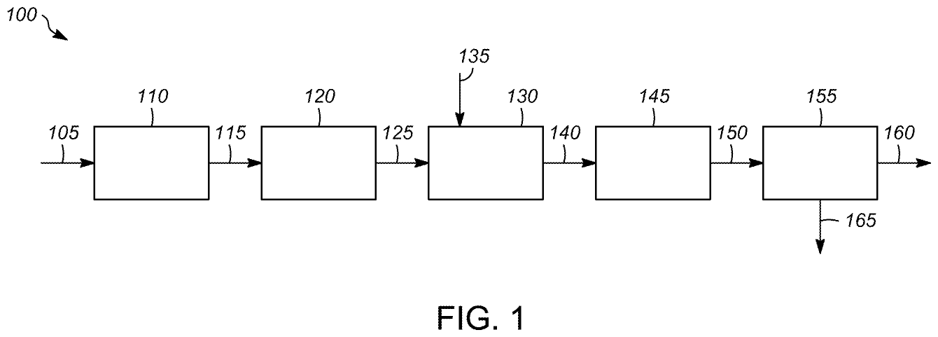

FIG. 1 illustrates on embodiment of a furfural process 100. The biomass feed 105 is sent to an optional conditioning unit 110. The conditioning unit 110 may be needed to obtain a moisture content in the desired range. If the moisture content in the biomass feed to too high, the conditioning unit 110 will typically be a dryer. If the moisture content is too low, it can be injected with steam, soaked in water, or placed in a humidifier, or other suitable treatment. If the moisture content of the biomass feed is acceptable, then the conditioning unit 110 is not needed.

The conditioned feed 115 is sent to a grinder 120 to reduce the particle size. The incoming biomass feed 105 typically screened through a three inch US screen. The biomass feed is ground to an average particle size in the range of 0.02 mm to 10 mm with 5 wt % or less having a particle size of 75 microns or less. The particles typically have an aspect ratio of 0.4 or more.

The ground biomass feed 125 may be sent to an optional collection tank 130 where it is blanketed with an inert gas 135. The inert gas has an oxidant or oxygen content of 5 mol % or less. Alternatively, the collection tank 130 may be omitted. In this case, the inert gas will be added to the transfer line to the reactor.

The blanketed ground biomass feed 140 is sent to the reaction zone 145 where it is reacted in the presence of water, a water-immiscible organic solvent, and an acid catalyst. The reaction zone 145 may comprise one or more reactors.

The reaction product mixture 150 is separated in a separation zone 155 into the furfural product stream 160 and a solvent stream 165.

### EXAMPLES

### Example 1

Corn cobs were obtained from the AGSCO company in Warrenville, IL. The C5 sugar content of AGSCO corn cobs is 34.4% (31.7% Xylan, 2.7% Arabinan). Since every C5 sugar molecule can convert into one molecule of furfural, the theoretical maximum yield of furfural is easily calculated once the C5 sugar content is known. Molecular weights for furfural and xylose/arabinose are 96.08 and 150.13 g/mol, respectively. Therefore, the maximum mass yield of furfural from corn cobs is approximately 22% of corn cob starting mass.

$$\begin{matrix}Theoretical grams of \\ furfural from corn cob\end{matrix} = \frac{\begin{matrix}1 gram of corn cob \times 34.4 \% C 5 sugars \times \\ Molecular weight of Furfural\end{matrix}}{Molecular weight of Xylose} = 0.22 grams of furfural$$

### Example 2: 1-Step Hydrolysis

Testing was conducted in a 300 cc Hastelloy Parr autoclave. Ground corn cob was added to the reactor vessel, followed by 100 ml of deionized (DI) water, then 7% formic acid, and finally 100 ml of toluene as the extracting solvent. The amount of corn cob was 20 grams, the reaction temperature was 170° C., and the reaction time was 2 hours. After the reaction, the contents of the reactor were removed and either centrifuged or filtered to separate the aqueous, organic, and solid phases. Samples of the aqueous and organic phases were filtered through a 0.2 μm PVDF filter for analysis. Sugars and organic acids were measured by liquid chromatography (for example, as shown in ASTM E1758-01) in the aqueous phase only. Furans were analyzed in both the aqueous phase by liquid chromatography and the organic phase by gas chromatography (for example, as shown in ASTM D5769).

### Conversion of Biomass to Furfural Using Sulfuric Acid

| Solvent | Feed | Observed Mass Yield % |
|---|---|---|
| Toluene | Corn Cobs | 16.8 |

### Example 3: 2-Step Hydrolysis

The same 75-cc autoclave from Example 2 was loaded with 4 wt. % ground corn cob feedstock, 7% formic acid, and 40 mL of DI water solution. The reactor was operated at a temperature of 120° C. for 6 hours. After that time, the biomass was removed from the reactor, but the product aqueous portion and new water and toluene solutions (aqueous:toluene weight ratio=1.3:1) were added back into the reactor.

The second phase reaction was conducted at 170° C. for a shorter period of 2 hours. The products were recovered from the second reaction for analysis. Under these conditions, there was 100% conversion of the hydrolyzed xylose, and 18.3% mass yield of furfural from biomass C5 sugar was achieved. The amount of levulinic acid and 5-hydroxymethylfurfural generated was less than 5 mol. % of the furfural.

### Specific Embodiments

While the following is described in conjunction with specific embodiments, it will be understood that this description is intended to illustrate and not limit the scope of the preceding description and the appended claims.

A first embodiment of the invention is a process for producing furfural or derivatives of furfural or both comprising providing a biomass feed having a moisture content of 25 wt % or less; grinding the biomass feed to form a ground biomass feed having a particle size in a range of 0.02 mm to 10 mm and 5 wt % or less having the particle size of 75 microns or less, and an average aspect ratio of 0.4 or more; blanketing the ground biomass feed with an inert gas resulting in an oxidant or oxygen content of 5 mol % or less; reacting the ground biomass feed in a reaction zone comprising a reactor in the presence of a solvent, and an acid catalyst to form a reaction product mixture comprising the furfural, the derivatives of furfural, or both wherein the solvent comprises water, and optionally a water immiscible solvent; and separating the furfural, the derivatives of furfural, or both from the reaction product mixture. An embodiment of the invention is one, any or all of prior embodiments in this paragraph up through the first embodiment in this paragraph wherein blanketing the ground biomass feed with the inert gas comprises blanketing the ground biomass feed in a collection vessel, and further comprising transferring the blanketed ground biomass feed to the reactor. An embodiment of the invention is one, any or all of prior embodiments in this paragraph up through the first embodiment in this paragraph wherein the moisture content of the biomass feed is 15 wt % or less. An embodiment of the invention is one, any or all of prior embodiments in this paragraph up through the first embodiment in this paragraph further comprising conditioning the biomass feed to obtain the moisture content of 25 wt % or less, An embodiment of the invention is one, any or all of prior embodiments in this paragraph up through the first embodiment in this paragraph wherein conditioning the biomass feed comprises drying the biomass feed. An embodiment of the invention is one, any or all of prior embodiments in this paragraph up through the first embodiment in this paragraph wherein the ground biomass feed has 1 wt % or less of particles having the size of 75 microns or less. An embodiment of the invention is one, any or all of prior embodiments in this paragraph up through the first embodiment in this paragraph wherein the oxygen content is 1 mol % or less. An embodiment of the invention is one, any or all of prior embodiments in this paragraph up through the first embodiment in this paragraph wherein the acid catalyst comprises a mineral acid, a solid acid, an organic acid, or combinations thereof. An embodiment of the invention is one, any or all of prior embodiments in this paragraph up through the first embodiment in this paragraph wherein the water-immiscible organic solvent is present and comprises benzene, toluene, a xylene, tetralin, an alkyl tetralin, an alkyl naphthalene, an aromatic alcohol, a dialkyl ketone, an alkyl phenol, or combinations thereof. An embodiment of the invention is one, any or all of prior embodiments in this paragraph up through the first embodiment in this paragraph wherein reacting the ground biomass feed comprises a hydrolysis reaction and a dehydration reaction. An embodiment of the invention is one, any or all of prior embodiments in this paragraph up through the first embodiment in this paragraph wherein there are two or more reactors. An embodiment of the invention is one, any or all of prior embodiments in this paragraph up through the first embodiment in this paragraph wherein the furfural derivatives comprise 5-hydroxymethylfurfural, 5-halogen methylfurfural wherein the halogen comprises a chloride, a bromide, a fluoride, or an iodide moiety, or combinations thereof.

A second embodiment of the invention is a process for producing furfural, derivatives of furfural, or both comprising providing a biomass feed having a moisture content of 5 to 15 wt %; grinding the biomass feed to form a ground biomass feed having an average particle size in a range of 0.02 mm to 10 mm and 5 wt % or less having a particle size of 75 microns or less, and an average aspect ratio of 0.4 or more; blanketing the ground biomass feed with an inert gas having an oxidant or oxygen content of 5 mol % or less; transferring the blanketed ground biomass feed to a reaction zone comprising a reactor; reacting the ground biomass feed in the reactor in the presence of a solvent, and an acid catalyst to form a reaction product mixture comprising the furfural, the derivatives of furfural, or both, wherein the solvent comprises water, and optionally a water immiscible solvent; and separating the furfural, the derivatives of furfural, or both from the reaction product mixture. An embodiment of the invention is one, any or all of prior embodiments in this paragraph up through the second embodiment in this paragraph further comprising drying the biomass feed to obtain the biomass feed having the moisture content of 5 to 15 wt %. An embodiment of the invention is one, any or all of prior embodiments in this paragraph up through the second embodiment in this paragraph wherein the ground biomass feed has 1 wt % or less of particles having the size of 75 microns or less. An embodiment of the invention is one, any or all of prior embodiments in this paragraph up through the second embodiment in this paragraph wherein the oxygen content is 1 mol % or less. An embodiment of the invention is one, any or all of prior embodiments in this paragraph up through the second embodiment in this paragraph wherein the acid catalyst comprises a mineral acid, a solid acid, an organic acid, or combinations thereof. An embodiment of the invention is one, any or all of prior embodiments in this paragraph up through the second embodiment in this paragraph wherein the water-immiscible organic solvent is present and comprises benzene, toluene, a xylene, tetralin, an alkyl tetralin, an alkyl naphthalene, an aromatic alcohol, a dialkyl ketone, an alkyl phenol, or combinations thereof. An embodiment of the invention is one, any or all of prior embodiments in this paragraph up through the second embodiment in this paragraph wherein reacting the ground biomass feed comprises a hydrolysis reaction and a dehydration reaction. An embodiment of the invention is one, any or all of prior embodiments in this paragraph up through the second embodiment in this paragraph wherein there are two or more reactors.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for producing furfural, or derivatives of furfural, or both comprising:
providing a biomass feed having a moisture content of 25 wt % or less;
grinding the biomass feed to form a ground biomass feed having a particle size in a range of 0.02 mm to 10 mm and 5 wt % or less having the particle size of 75 microns or less, and an average aspect ratio of 0.4 or more;
blanketing the ground biomass feed with an inert gas resulting in an oxidant or oxygen content of 5 mol % or less;
reacting the ground biomass feed in a reaction zone comprising a reactor in the presence of a solvent, and an acid catalyst to form a reaction product mixture comprising the furfural, the derivatives of furfural, or both wherein the solvent comprises water, and optionally a water immiscible solvent; and
separating the furfural, the derivatives of furfural, or both from the reaction product mixture.

2. The process of claim 1 wherein blanketing the ground biomass feed with the inert gas comprises blanketing the ground biomass feed in a collection vessel, and further comprising:
transferring the blanketed ground biomass feed to the reactor.

3. The process of claim 1 wherein the moisture content of the biomass feed is 15 wt % or less.

4. The process of claim 1 further comprising:
conditioning the biomass feed to obtain the moisture content of 25 wt % or less.

5. The process of claim 4 wherein conditioning the biomass feed comprises drying the biomass feed.

6. The process of claim 1 wherein the ground biomass feed has 1 wt % or less of particles having the size of 75 microns or less.

7. The process of claim 1 wherein the oxygen content is 1 mol % or less.

8. The process of claim 1 wherein the acid catalyst comprises a mineral acid, a solid acid, an organic acid, or combinations thereof.

9. The process of claim 1 wherein the water-immiscible organic solvent is present and comprises benzene, toluene, a xylene, tetralin, an alkyl tetralin, an alkyl naphthalene, an aromatic alcohol, a dialkyl ketone, an alkyl phenol, or combinations thereof.

10. The process of claim 1 wherein reacting the ground biomass feed comprises a hydrolysis reaction and a dehydration reaction.

11. The process of claim 1 wherein there are two or more reactors.

12. A process for producing furfural, derivatives of furfural, or both comprising:
providing a biomass feed having a moisture content of 5 to 15 wt %;
grinding the biomass feed to form a ground biomass feed having an average particle size in a range of 0.02 mm to 10 mm and 5 wt % or less having a particle size of 75 microns or less, and an average aspect ratio of 0.4 or more;
blanketing the ground biomass feed with an inert gas having an oxidant or oxygen content of 5 mol % or less;
transferring the blanketed ground biomass feed to a reaction zone comprising a reactor;
reacting the ground biomass feed in the reactor in the presence of a solvent, and an acid catalyst to form a reaction product mixture comprising the furfural, the derivatives of furfural, or both wherein the solvent comprises water, and optionally a water immiscible solvent; and
separating the furfural, the derivatives of furfural, or both from the reaction product mixture.

13. The process of claim 12 further comprising:
drying the biomass feed to obtain the biomass feed having the moisture content of 5 to 15 wt %.

14. The process of claim 12 wherein the ground biomass feed has 1 wt % or less of particles having the size of 75 microns or less.

15. The process of claim 12 wherein the oxygen content is 1 mol % or less.

16. The process of claim 12 wherein the acid catalyst comprises a mineral acid, a solid acid, an organic acid, or combinations thereof.

17. The process of claim 12 wherein the water-immiscible organic solvent is present and comprises benzene, toluene, a xylene, tetralin, an alkyl tetralin, an alkyl naphthalene, an aromatic alcohol, a dialkyl ketone, an alkyl phenol, or combinations thereof.

18. The process of claim 12 wherein reacting the ground biomass feed comprises a hydrolysis reaction and a dehydration reaction.

19. The process of claim 12 wherein there are two or more reactors.

20. The process of claim 1, wherein the furfural derivatives comprise 5-hydroxymethylfurfural, 5-halogen methylfurfural wherein the halogen comprises a chloride, a bromide, a fluoride, or an iodide moiety, or combinations thereof.
